(19) United States
(12) Patent Application Publication
(10) Pub. No.: US 2026/0027214 A1
(43) Pub. Date: Jan. 29, 2026

(54) PHARMACEUTICAL COMPOSITION FOR TREATING OR PREVENTING INFLAMMATION CAUSED BY ACANTHAMOEBA

(71) Applicants: University of Tsukuba, Tsukuba-shi, Ibaraki (JP); WAKO FILTER TECHNOLOGY CO., LTD., Chiyoda-ku, Tokyo (JP)

(72) Inventors: Yuichi KAJI, Tsukuba-shi, Ibaraki (JP); Shoko TAKAHASHI, Tsukuba-shi, Ibaraki (JP); Hiroki KAGESHIMA, Bando-shi, Ibaraki (JP); Shouichirou HIRANO, Bando-shi, Ibaraki (JP)

(73) Assignees: University of Tsukuba, Tsukuba-shi, Ibaraki (JP); WAKO FILTER TECHNOLOGY CO., LTD., Chiyoda-ku, Tokyo (JP)

(21) Appl. No.: 18/995,592

(22) PCT Filed: Jul. 20, 2023
(86) PCT No.: PCT/JP2023/026529

§ 371 Date: Jan. 16, 2025

(30) Foreign Application Priority Data
Aug. 23, 2022 (JP) 2022-132244

(51) Int. Cl. A61K 47/55; C12N 9/36

(52) U.S. Cl. CPC A61K 47/55; C12N 9/2462; C12Y 302/01017

(57) ABSTRACT

A pharmaceutical composition for treating or preventing inflammation caused by Acanthamoeba that contains a composite formed by joining lysozyme and chitosan is described. Also described are an antiprotozoal composition contains a composite formed by joining lysozyme and chitosan, an anti-attachment agent for Acanthamoeba that contains a composite formed by joining lysozyme and chitosan.

### TECHNICAL FIELD

The present invention relates to pharmaceutical compositions and the like for treating or preventing inflammation caused by Acanthamoeba.

### BACKGROUND ART

Acanthamoeba is a type of amoeba protozoan (protist) that is a microorganism commonly found in soil and other environments. Acanthamoeba infects the eye, particularly the cornea, causing corneal inflammation. Inflammation caused by Acanthamoeba is especially common among contact lens wearers; for example, it can adhere to contact lens cases through tap water, proliferate within the case, and cause corneal infection through contact lenses.

Various amebicidal agents have been developed as countermeasures against Acanthamoeba, with biguanide-based germicides (Patent Literatures 1-3) being notable examples. However, few compositions other than biguanide-based germicides are known to exhibit strong anti-amoebic effects.

### CITATION LIST

### Patent Literatures

Patent Literature 1: International Publication No. 02/4963 Pamphlet
Patent Literature 2: International Publication No. WO 2013/154016 Pamphlet
Patent Literature 3: International Publication No. WO 2014/192068 Pamphlet

### SUMMARY OF INVENTION

The present invention aims to provide a novel pharmaceutical composition for treating or preventing inflammation caused by Acanthamoeba. The present invention also aims to provide an antiprotozoal composition that exhibits effective therapeutic and preventive effects against protozoal infections including Acanthamoeba, methods for treating and preventing protozoal infections including Acanthamoeba, and methods for using such pharmaceutical compositions in the manufacture of antiprotozoal compositions.

### Means for Solution of the Problems

The inventors of the present invention used a complex of lysozyme and chitosan, both of which are safe enough to be used as food additives, to evaluate the antiprotozoal and anti-adhesion effects against Acanthamoeba, and they have newly discovered as a result that it exhibits excellent antiprotozoal and anti-adhesion effects against Acanthamoeba, leading to the present invention. More specifically, the present invention may comprise the following aspects:
[1] A pharmaceutical composition for treating or preventing inflammation caused by Acanthamoeba, comprising a complex of lysozyme and chitosan bound together.
[2] The pharmaceutical composition according to [1], wherein the chitosan is a water-soluble chitosan having a molecular weight of 1000 Da to 30,000 Da.
[3] The pharmaceutical composition according to [1] or [2], wherein the complex is contained in 0.01 μg/mL to 100,000 μg/mL in the pharmaceutical composition.
[4] The pharmaceutical composition according to [1] or [2], wherein the complex is contained in 0.0001 mass % to 5.0 mass % in the pharmaceutical composition.
[5] The pharmaceutical composition according to any one of [1] to [4], further comprising an additional active ingredient selected from the group consisting of hydrogen peroxide solution, povidone iodine, polidronium chloride, and polyhexanide hydrochloride.
[6] The pharmaceutical composition according to any one of [1] to [5], wherein the inflammation caused by Acanthamoeba is dermatitis or keratitis.
[7] The pharmaceutical composition according to any one of [1] to [6], wherein the treatment or prevention of inflammation caused by Acanthamoeba is achieved through inhibition of Acanthamoeba adhesion.
[8] Use of a complex of lysozyme and chitosan bound together in the manufacture of a pharmaceutical composition for treating or preventing inflammation caused by Acanthamoeba.
[9] An antiprotozoal composition comprising a complex of lysozyme and chitosan bound together.
[10] The antiprotozoal composition according to [9], wherein the protozoan is Acanthamoeba.
[11] An anti-adhesion agent for Acanthamoeba comprising a complex of lysozyme and chitosan bound together.
[12] A method of preventing adhesion of Acanthamoeba to a material surface, comprising applying a complex of lysozyme and chitosan bound together to the material surface.
[13] The method for preventing adhesion of Acanthamoeba according to [12], wherein the material is selected from the group consisting of polyethylene, polypropylene, polyurethane, and polycarbonate.

### Advantageous Effects of Invention

The present invention exhibits effects of removing, inhibiting growth of, or killing Acanthamoeba protozoa, as well as suppressing their proliferation. In particular, the complex of lysozyme and chitosan bound together in the present invention not only inhibits the proliferation of Acanthamoeba but also exhibits significant lethal effects. Therefore, the present invention can cure and/or prevent Acanthamoeba infections. Moreover, lysozyme is widely used as a highly safe, naturally-derived food additive, and this antiprotozoal composition using a complex of lysozyme and chitosan can reassure patients who use the complex while reducing their burden.

### BRIEF DESCRIPTION OF DRAWINGS

FIG. 1 is a schematic diagram of the reaction for synthesizing a complex of lysozyme and chitosan bound together.

FIG. 2 is a biological microscope photograph of Acanthamoeba observed during measurement of “Total number of adhered Acanthamoeba” in Table 1.

FIG. 3 is a biological microscope photograph showing dead Acanthamoeba stained dark with trypan blue during measurement of “Number of dead Acanthamoeba” in Table 1.

FIG. 4 is a graph comparing the total number of adhered Acanthamoeba when using solutions of lysozyme and chitosan mixture, lysozyme alone, and chitosan alone in Tables 2-4 with the case using the complex of lysozyme and chitosan bound together in Table 1.

FIG. 5 is a graph comparing the mortality rate of Acanthamoeba when using solutions of lysozyme and chitosan mixture, lysozyme alone, and chitosan alone in Tables 2-4 with the case using the complex of lysozyme and chitosan bound together in Table 1.

FIG. 6 is a graph showing the total number of adhered Acanthamoeba when using the lysozyme-chitosan complex (LYZOX (registered trademark)) in combination with additional active ingredients in Table 5.

FIG. 7 is a graph showing the mortality rate of Acanthamoeba when using the lysozyme-chitosan complex (LYZOX (registered trademark)) in combination with additional active ingredients in Table 5.

### DESCRIPTION OF EMBODIMENTS

A detailed description of an embodiment of the present invention is provided below. Note that the preferred embodiments and more preferred embodiments exemplified below may be combined appropriately regardless of expressions such as “preferred” or “more preferred.” Additionally, the numerical ranges provided are exemplary, and ranges appropriately combining the upper and lower limits of each range as well as numerical values from examples may be preferably used. Furthermore, terms such as “containing” or “comprising” may be interpreted as “consisting essentially of” or “consisting only of.”

### <Pharmaceutical Composition>

The present invention relates to a pharmaceutical composition for treating or preventing inflammation caused by Acanthamoeba, comprising a complex of lysozyme and chitosan bound together. The pharmaceutical composition is described in detail below.

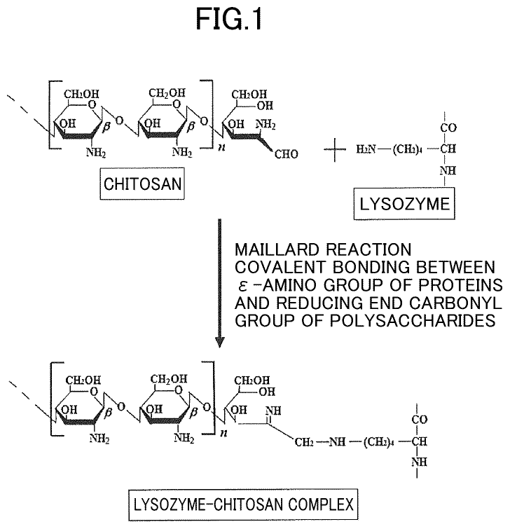

The “complex of lysozyme and chitosan bound together” refers to a complex in which lysozyme and chitosan are bound together, for example, through a Maillard reaction or other means (see FIG. 1). When lysozyme and water-soluble chitosan are bound through a Maillard reaction, most or all of the antigenic structures in lysozyme are masked; therefore, the lysozyme-chitosan complex has the characteristic of being less likely to cause allergies when ingested by humans. The complex can also be obtained by covalently binding lysozyme and chitosan using a crosslinking agent.

Here, “lysozyme” is an enzyme that hydrolyzes mucopolysaccharides, and lysozyme derived from chicken or human sources can be preferably used.

The upper limit of the molecular weight of lysozyme may be, for example, 30,000 Da or less, more preferably 25,000 Da or less, 20,000 Da or less, 18,000 Da or less, or 15,000 Da or less. While there is no particular restriction on the lower limit of the molecular weight of lysozyme, it may be, for example, 1000 Da or more, preferably 5,000 Da or more, 10,000 Da or more, or 12,000 Da or more. The range of the molecular weight of the lysozyme may be between any of the above upper and lower limits, but may be, for example, 1000 Da to 30,000 Da, preferably 5,000 Da to 20,000 Da, more preferably 10,000 Da to 15,000 Da.

“Chitosan” is poly-β1→4-glucosamine represented by the following chemical formula (I) ((C₆H₁₁NO₄)ₙ, CAS Registry Number 9012-76-4).

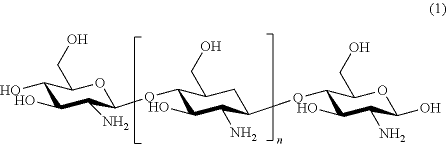

The chitosan is water-soluble. The upper limit of the molecular weight of chitosan may be, for example, 30,000 Da or less, more preferably 20,000 Da or less, 15,000 Da or less, 10,000 Da or less, or 7,000 Da or less. While there is no particular restriction on the lower limit of the molecular weight of chitosan, it may be, for example, 300 Da or more, preferably 500 Da or more, 1,000 Da or more, or 3,000 Da or more. The range of the molecular weight of the chitosan may be between any of the above upper and lower limits, but may be, for example, 300 Da to 30,000 Da, preferably 500 Da to 15,000 Da, more preferably 1,000 Da to 10,000 Da, particularly preferably 3,000 Da to 7,000 Da. From the perspective of antiprotozoal activity, higher molecular weight chitosan is advantageous, while from the perspective of ease of manufacture, lower molecular weight chitosan is advantageous due to better solubility and stability.

The term chitosan as used here includes, in addition to the above chitosan, chitosan oligosaccharides and glucosamine. Chitosan oligosaccharides refer to several D-glucosamine units linked together as shown in formula (I), meaning low molecular weight chitosan or hydrolysates of chitosan in the narrow sense obtained by hydrochloric acid or enzymatic hydrolysis.

Examples of the above crosslinking agents include amine-reactive crosslinking agents (for example, alkoxyamines), carbonyl-reactive crosslinking agents (for example, hydrazine compounds), and sulfhydryl-reactive crosslinking agents.

The mass ratio of lysozyme/chitosan is appropriately, for example, 99/1 to 1/99, preferably 90/10 to 10/90, more preferably 80/20 to 20/80, further preferably 60/40 to 40/60, particularly preferably 50/50.

As for specific methods for manufacturing the complex of lysozyme and chitosan bound together, the following method can be cited as an example. First, lysozyme and chitosan in the above mass ratio are mixed and dissolved in water, and the solution is prepared such that the total mass of lysozyme and chitosan in the obtained aqueous solution is 5 to 30 mass %. The obtained aqueous solution is freeze-dried to form a powder. The obtained powder is subjected to Maillard reaction under conditions of, for example, temperature of 50 to 80° C., preferably 55 to 65° C., and relative humidity of, for example, 50 to 80%, preferably 60 to 70%, for a period of, for example, 2 to 20 days, more preferably 7 to 14 days, thereby manufacturing the complex of lysozyme and chitosan bound together of the present invention.

Formation of the lysozyme-chitosan complex in this embodiment can be confirmed by various known methods; for example, the formation of a high molecular weight substance, which is a protein-chitosan complex, can be confirmed by staining a plate obtained through SDS (Sodium dodecyl sulfate) or SDS-PAGE (Sodium dodecyl sulfate-polyacrylamide) polyacrylamide gel electrophoresis.

“Acanthamoeba” (Acanthamoeba polyphaga) is a protozoan and a type of amoeba classified among protozoa, particularly within the Rhizopoda class, and exists in environments such as soil and freshwater. As mentioned above, Acanthamoeba infects the skin and eyes, particularly the cornea, causing skin inflammation and corneal inflammation. Inflammation caused by Acanthamoeba is especially common among contact lens wearers; for example, Acanthamoeba adheres to contact lens cases through tap water, proliferates within the case, and causes corneal infection through contact lens solution or contact lenses, leading to inflammation. Types of inflammation include dermatitis and corneal keratitis. In particular, the present invention is expected to be effective against eye disorders such as corneal hyperemia, corneal opacity, radial keratoneuritis, disciform corneal ulcer, scleritis, and endophthalmitis.

The present invention exhibits “antiprotozoal” activity against “Acanthamoeba.” “Protozoa” includes amoebae such as Acanthamoeba. Here, antiprotozoal activity broadly encompasses prevention, removal, killing, inhibition of protozoa, and all conditions where the number of protozoa remains equal or decreases, including at least preventing protozoan proliferation. Furthermore, removal of protozoa encompasses all aspects of eliminating protozoa, including killing protozoa. Killing protozoa means causing death to at least some of the protozoa. Therefore, in the present invention, antiprotozoal composition includes compositions for protozoan removal, protozoacidal compositions, protozoan removal agents, protozoacidal agents, and antiprotozoal agents.

In particular, the present invention can be used as an anti-adhesion agent to prevent Acanthamoeba from adhering to materials such as contact lens cases. While Acanthamoeba tends to adhere to plastic surfaces such as contact lens cases, the presence of the present invention in contact lens cleaning solution and the like can inhibit such adhesion of Acanthamoeba. The aforementioned treatment or prevention of inflammation caused by Acanthamoeba can also be achieved through inhibition of Acanthamoeba adhesion. The anti-adhesive effect of the present invention against Acanthamoeba is effective on various material surfaces, including: polypropylene used in contact lens cases as well as polyethylene, polyurethane, polycarbonate, polyvinyl polymers, melamine resin, urea resin, epoxy resin, acrylic resin, silicone, elastomers, rubber, and other polymeric resin compositions used in contact lens cases, cleaning solution bottles, blister packs for containing unworn contact lenses, toys, and the like; contact lens materials such as hydroxyethyl methacrylate, silicone hydrogel, polymethyl methacrylate, and glass; and various materials such as paper, metal, cloth, wood, ceramics, and leather. Here, “materials” may be defined to exclude human or animal bodies and parts thereof.

The present invention is also effective in treating and preventing inflammation caused by “Acanthamoeba.” Here, “treatment” includes completely curing the inflammation targeted by the present invention, as well as suppressing inflammation to reduce its severity. “Prevention” includes cases where there is no history of inflammation targeted by the present invention, as well as preventing recurrence after the inflammation targeted by the present invention has been cured.

The pharmaceutical composition of the present embodiment may optionally include one or more additional active ingredients besides the above complex. Additional active ingredients include those that act as active ingredients themselves, as well as those that do not act as active ingredients alone but exhibit effects when used in combination with the complex, which is the active ingredient of the present invention (auxiliary agents). Examples of additional active ingredients include: biguanide-based germicides, hydrogen peroxide solution, povidone iodine (complex of 1-vinyl-2-pyrrolidone polymer and iodine), polidronium chloride (Polyquad (registered trademark)), polyhexanide hydrochloride (PHMB), terpene alcohols, fatty acids, and/or salts thereof. Adding one or more of these additional active ingredients can achieve additive and/or synergistic effects in combination with the complex of the present invention. Particularly preferred are hydrogen peroxide solution, povidone iodine (complex of 1-vinyl-2-pyrrolidone polymer and iodine), polidronium chloride (Polyquad (registered trademark)), and polyhexanide hydrochloride (PHMB).

Examples of terpene alcohols include terpinen-4-ol, hinokitiol, geraniol, and menthol. Examples of fatty acids include fatty acids with 8 to 12 carbon atoms, preferably decanoic acid (capric acid) or lauric acid. Examples of fatty acid salts include sodium salts, potassium salts, magnesium salts, and calcium salts. Additionally, other substances known to be effective against inflammation caused by Acanthamoeba may be added as additional active ingredients, such as polyhexanide hydrochloride, lactoferrin, boric acid and/or its salts, caspofungin and/or its salts, voriconazole, miconazole, econazole, sulconazole, fluconazole, quaternary ammonium-based disinfectants, and chitinase.

The pharmaceutical composition of the present invention may optionally include one or more other components besides the above complex. Other components may include additives such as excipients, binders, emulsifiers, solvents, adhesives, disintegrants, thickeners, lubricants, colorants, flow agents, and moisturizing agents.

Examples of moisturizing agents include glycerin, butylene glycol, collagen, hyaluronic acid, and ceramide.

Examples of binders include cellulose, methyl cellulose, hydroxyethyl cellulose, and sodium carboxymethyl cellulose.

Examples of emulsifiers include lecithin, polyethylene glycol (PG), PG-hydrogenated castor oil, glycerin fatty acid esters, sorbitan fatty acid esters, and ceteth.

Examples of solvents include water, ethanol, 1-butanol, 2-butanol, 1-propanol, 2-propanol, and 1-pentanol.

The lysozyme-chitosan complex of the present invention is appropriately contained in concentrations of, for example, 0.01 μg/mL or more, 0.05 μg/mL or more, 0.1 μg/mL or more, 0.5 μg/mL or more, 1 μg/mL or more, 10 μg/mL or more, 50 μg/mL or more, or 100 μg/mL or more per mL of the pharmaceutical composition. Additionally, the lysozyme-chitosan complex is appropriately contained in concentrations of, for example, 100,000 μg/mL or less, 50,000 μg/mL or less, 10,000 μg/mL or less, 5,000 μg/mL or less, or 1,000 μg/mL or less per mL of the pharmaceutical composition. The preferred range of the content of the lysozyme-chitosan complex may be a range appropriately combining these lower and upper limits, but may be, for example, 0.1 μg/mL to 10,000 μg/mL, preferably 0.5 μg/mL to 5,000 μg/mL or 50 μg/mL to 5,000 μg/mL, and more preferably 1 μg/mL to 1,000 μg/mL or 100 μg/mL to 1,000 μg/mL.

The lysozyme-chitosan complex of the present invention is also appropriately contained in concentrations of, for example, 0.0001 mass % or more, 0.001 mass % or more, 0.005 mass % or more, 0.01 mass % or more, 0.05 mass % or more, 0.1 mass % or more, 0.5 mass % or more, or 1 mass % or more relative to the total mass of the pharmaceutical composition. Additionally, the lysozyme-chitosan complex is appropriately contained in concentrations of, for example, 5.0 mass % or less, 1 mass % or less, 0.5 mass % or less, 0.1 mass % or less, 0.05 mass % or less, 0.01 mass % or less, or 0.005 mass % or less relative to the total mass of the pharmaceutical composition. The preferred range of the content of the lysozyme-chitosan complex may be a range appropriately combining these lower and upper limits, but may be, for example, 0.001 mass % to 5.0 mass %, preferably 0.005 mass % to 1.0 mass %, more preferably 0.01 mass % to 0.5 mass %.

Additionally, the additional active ingredients are appropriately contained in concentrations of, for example, 0.00001 mass % to 20 mass %, preferably 0.00005 mass % to 10 mass %, more preferably 0.0001 mass % to 5 mass %, further preferably 0.0005 mass % to 3 mass %, particularly preferably 0.001 mass % to 1 mass % in the pharmaceutical composition. The mass ratio of the lysozyme-chitosan complex of the present invention to the additional active ingredients is appropriately, for example, 99/1 to 1/99, preferably 90/10 to 10/90, more preferably 80/20 to 20/80, further preferably 60/40 to 40/60, particularly preferably 50/50.

Furthermore, while other components must be appropriately adjusted for suitable contents depending on each component, they are appropriately contained in concentrations of, for example, 0.1 mass % to 90 mass %, preferably 1 mass % to 70 mass %, more preferably 10 mass % to 50 mass %. The pharmaceutical composition of the present invention may consist mostly of solvents such as water, and may appropriately contain, for example, 95 mass % or more, preferably 98 mass % or more, more preferably 99 mass % or more, further preferably 99.5 mass % or more of solvent in the pharmaceutical composition.

### <Antiprotozoal Composition>

The present invention further relates to an antiprotozoal composition comprising a complex of lysozyme and chitosan bound together. Details regarding the content of the complex of lysozyme and chitosan bound together that may be contained in the antiprotozoal composition, additional active ingredients, other components, etc., are all as described above for the pharmaceutical composition.

### <Anti-Adhesion Agent>

The present invention further relates to an anti-adhesion agent comprising a complex of lysozyme and chitosan bound together. Details regarding the content of the complex of lysozyme and chitosan bound together that may be contained in the anti-adhesion agent, additional active ingredients, other components, etc., are all as described above for the pharmaceutical composition. Without wishing to be bound by theory, this anti-adhesive property is thought to be achieved through the lysozyme-chitosan complex binding to Acanthamoeba through electrostatic mechanisms, thereby inhibiting Acanthamoeba adhesion to surfaces such as plastic. Since the lysozyme-chitosan complex is positively charged while Acanthamoeba is negatively charged, it is thought that competition between Acanthamoeba adhesion to material surfaces and complex adhesion results in anti-adhesive action.

The anti-adhesion agent is typically included in contact lens cleaning solutions or storage solutions. The content of the anti-adhesion agent in cleaning solutions or storage solutions can be specified by the proportion of the complex of lysozyme and chitosan bound together contained in the anti-adhesion agent, and it is appropriate for the complex to be contained in concentrations of, for example, 0.001 μg/mL or more, 0.01 μg/mL or more, 0.1 μg/mL or more, 0.5 μg/mL or more, 1 μg/mL or more, 10 μg/mL or more, 50 μg/mL or more, or 100 μg/mL or more per mL of cleaning solution or storage solution. Additionally, the lysozyme-chitosan complex of the present invention is appropriately contained in concentrations of, for example, 100,000 μg/mL or less, 50,000 μg/mL or less, 10,000 μg/mL or less, 5,000 μg/mL, or 1,000 μg/mL or less per mL of cleaning solution or storage solution. The preferred range of the content of the lysozyme-chitosan complex may be a range appropriately combining these lower and upper limits, but may be, for example, 0.01 μg/mL to 100,000 μg/mL, preferably 0.5 μg/mL to 10,000 μg/mL, and more preferably 0.1 μg/mL to 1,000 μg/mL.

Here, contact lenses may be either soft contact lenses or hard contact lenses commonly used. Cleaning solutions or storage solutions may be physiological saline or purified water containing components such as surfactants, thickeners, and moisturizing agents typically used for cleaning or storing contact lenses, or may be storage solutions in blister packs containing new, unworn contact lenses.

### <Treatment Method, Prevention Method>

The present invention may include methods for treating or preventing inflammation caused by Acanthamoeba by administering to a subject a pharmaceutical composition comprising a complex of lysozyme and chitosan bound together. The treatment or prevention is directed toward, for example, treating or preventing infection, inflammation, and/or disease of the skin and eyes, particularly the cornea of the eye. Without wishing to be bound by theory, the therapeutic or preventive effects are the to be attributable to the aforementioned antiprotozoal and/or anti-adhesive properties of the complex of lysozyme and chitosan bound together.

Here, details of the pharmaceutical composition used in the treatment or prevention methods and various definitions are as described above, and they may include additional active ingredients and other aspects; all details regarding definitions of lysozyme and chitosan, contents and ratios of additional active ingredients and other components, etc., are as described above for the pharmaceutical composition.

Here, “subject” includes mammals such as cats, dogs, monkeys, cattle, and horses, in addition to humans.

### <Dosage Amount, Dosage Form, Administration Method>

While varying depending on the formulation, administration subject, administration route, target disease, symptoms, etc., of the pharmaceutical composition of the present invention, in the case of a spray formulation for spraying on human body parts such as the body, hands, or fingers, the daily dosage is desirably, for example, 0.1 to 20 mg/kg body weight, preferably 0.2 to 10 mg/kg body weight, further preferably 0.5 to 10 mg/kg body weight, administered once to several times per day (e.g., 2, 3, 4, or 8 times). This dosage amount can be preferably applied to other formulations besides spray formulations, such as creams explained below.

The pharmaceutical composition of the present invention can be administered either orally or parenterally, and formulation does not require any special technology; it can be formulated using commonly used technologies. Dosage forms include creams, ointments, patch agents, liquid preparations, spray preparations, gel preparations, injections, tablets, suppositories, capsules, granules, powders, eye drops, and ophthalmic ointments, with creams, ointments, patch agents, liquid preparations, and spray preparations being particularly preferred.

The pharmaceutical composition of the present invention can select appropriate administration methods according to the above dosage forms. The administration method may follow known administration methods; for example, in the case of cream, the pharmaceutical composition may be applied to the inflamed area at the above dosage amount.

### <Antiprotozoal Method, Usage Amount, Usage Form, Usage Method>

The present invention can, for example, prevent, remove, kill, and inhibit protozoa such as Acanthamoeba by using, on a target, an antiprotozoal composition comprising a complex of lysozyme and chitosan bound together. The meaning of antiprotozoal activity is as described above. When the target is mammals such as humans, the explanation of treatment methods, prevention methods, dosage amounts, dosage forms, and administration methods related to the above pharmaceutical composition can be applied to this antiprotozoal method. Even when the target is non-human, the usage amount, usage form, and usage method can be selected according to the treatment methods, prevention methods, dosage amounts, dosage forms, and administration methods related to the above pharmaceutical composition. For example, when the target is a contact lens case, the antiprotozoal composition may be distributed as a liquid preparation or spray preparation to the contact lens case.

### <Anti-Adhesion Method, Usage Amount, Usage Form, Usage Method>

The present invention performs prevention, removal, and other actions against the adhesion of protozoa such as Acanthamoeba by using, on a target, an anti-adhesion agent comprising a complex of lysozyme and chitosan bound together. The meaning of anti-adhesive properties and adhesion targets are as described above. The usage amount, usage form, and usage method of the anti-adhesion agent can apply the explanation of treatment methods, prevention methods, dosage amounts, dosage forms, and administration methods related to the above pharmaceutical composition. For example, when the anti-adhesion target is a contact lens case, the anti-adhesion agent may be mixed as a liquid preparation in contact lens cleaning solution or storage solution.

### <Use>

The present invention may also include the use of a complex of lysozyme and chitosan bound together in the manufacture of a pharmaceutical composition for treating or preventing inflammation caused by Acanthamoeba, an antiprotozoal composition, and an anti-adhesion agent, all comprising a complex of lysozyme and chitosan bound together.

In the manufacture of the pharmaceutical composition, antiprotozoal composition, and anti-adhesion agent, the complex of lysozyme and chitosan bound together may be optionally mixed with additional active ingredients and other components besides the complex to form the pharmaceutical composition, etc. Known methods can be used as mixing methods, but for example, in the case of liquid preparations, a solvent such as water is mixed with the complex of lysozyme and chitosan bound together and the above optional other components, and when necessary, an emulsifier is added and mixed to form a dispersion or emulsion, thereby preparing the liquid preparation.

These pharmaceutical composition, antiprotozoal composition, and anti-adhesion agent can be used as cleaning water for nebulizers. By spraying the pharmaceutical composition, antiprotozoal composition, and anti-adhesion agent to necessary locations, proliferation and adhesion of protozoa such as Acanthamoeba can be inhibited, and the protozoa can be killed.

Additionally, the pharmaceutical composition, antiprotozoal composition, and anti-adhesion agent of this embodiment exhibit, for example, antiprotozoal effects and anti-adhesive effects against Acanthamoeba, as well as therapeutic, suppressive, and preventive effects against inflammation caused by these.

The following examples further illustrate the present invention in detail, but the present invention is not limited to these examples. In this specification and drawings, the complex of lysozyme and chitosan bound together may also be referred to as lysozyme-chitosan complex, or LYZOX (which is a registered trademark of Wako Filter Technology Co., Ltd.).

### EXAMPLES

### [Sample Preparation]

### Lysozyme-Chitosan Complex (LYZOX (Registered Trademark)) Solution

As a solution of the complex of lysozyme and chitosan bound together (lysozyme-chitosan complex), commercially available LYZOX (registered trademark, manufactured by Wako Filter Technology Co., Ltd.) was used. Commercial LYZOX (registered trademark) is a powder containing chicken-derived lysozyme (approximately 14,000 Da) and water-soluble chitosan (chitosan oligosaccharide) of approximately 5,000 Da in a mass ratio of 1:1. Specifically, the above lysozyme and water-soluble chitosan were dissolved in water, then freeze-dried to form a powder, and further subjected to Maillard reaction under conditions with sufficient temperature, humidity, and duration for the Maillard reaction to completely finish, thereby obtaining the lysozyme-chitosan complex (LYZOX (registered trademark)) used in this invention. This lysozyme-chitosan complex (LYZOX (registered trademark)) was mixed with sterilized water to achieve the desired concentration of the complex in each experiment to prepare the target lysozyme-chitosan complex (LYZOX (registered trademark)) solution.

### Lysozyme Alone Solution

The lysozyme alone solution was prepared by mixing egg white lysozyme with molecular weight of approximately 14300 (manufactured by Biocon (Japan) Ltd., Aichi Prefecture) with sterilized water to achieve the desired concentration of lysozyme in each experiment.

### Chitosan Alone Solution

The chitosan alone solution was prepared using chitosan oligosaccharide (Kimica Chitosan Oligosaccharide COS-A manufactured by Kimica Corporation) as chitosan, mixed with sterilized water to achieve the desired concentration of chitosan oligosaccharide in each experiment.

### Mixture Solution of Lysozyme and Chitosan

The mixture of lysozyme and chitosan was prepared by mixing the above lysozyme alone and chitosan alone in a mass ratio of 1:1, and further mixing with sterilized water to achieve the desired concentration of the mixture, lysozyme, and chitosan.

### Acanthamoeba Solution (Suspension)

An Acanthamoeba solution containing 1×10⁵ Acanthamoeba per mL of solution was prepared by dispersing Acanthamoeba (Acanthamoeba polyphaga, obtained from American Type Culture Collection (ATCC), 30872™) in phosphate-buffered saline as solvent.

### Test Pieces

The polypropylene test pieces used in the adhesion test described below were polypropylene from Johoku Co., Ltd., product number: PP_A3, cut to dimensions of 15 mm length×15 mm width×0.25 mm thickness from original dimensions of 320 mm length×440 mm width×0.25 mm thickness for each experiment.

Details of specific experimental examples are shown below.

### Experimental Example 1 (Adhesion Test and Cell Death Test)

To verify the anti-adhesive properties and antiprotozoal activity of the lysozyme-chitosan complex (LYZOX (registered trademark)), adhesion tests and cell death tests were conducted.

Specifically, the above-mentioned Acanthamoeba solution was added to the above-mentioned lysozyme-chitosan complex (LYZOX (registered trademark)) solution to prepare mixed solutions containing 0.1 μg, 1 μg, 10 μg, 100 μg, and 1000 μg of lysozyme-chitosan complex per 1 mL of mixed solution. Separately, a control solution with 0 μg/mL concentration containing no lysozyme-chitosan complex was prepared. The same sterilized water used in preparing the lysozyme-chitosan complex was used as dilution liquid for concentration adjustment. All six solutions contained 1×10⁵ Acanthamoeba per 1 mL of solution.

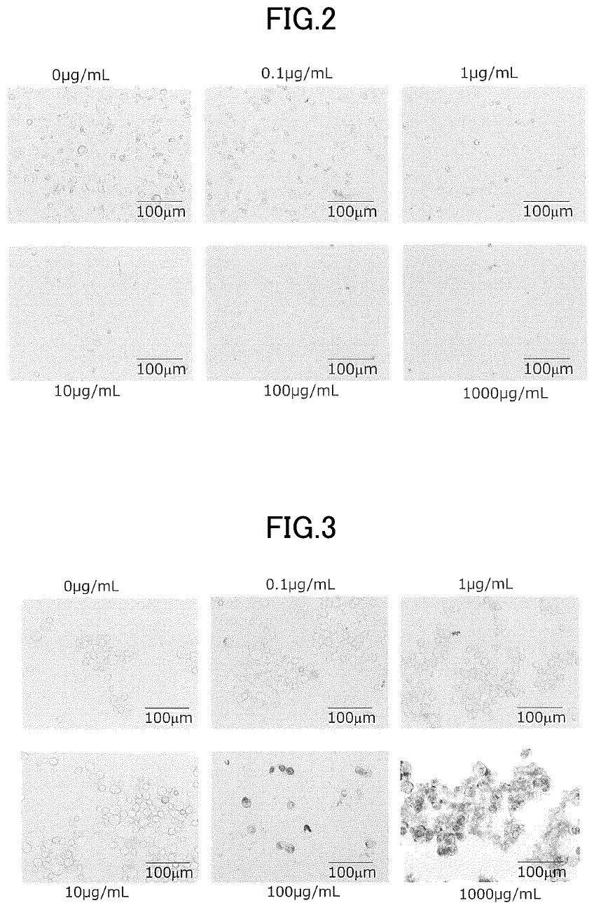

The above-mentioned polypropylene test pieces were immersed in the prepared mixed solutions and left standing; after 60 minutes of immersion, the test pieces were removed from the mixed solutions, washed with phosphate-buffered saline (PBS, obtained from FUJIFILM Wako Pure Chemical Corporation, product number: 166-23555), and dried. The surface of the obtained test pieces was observed under a biological microscope (obtained from Nikon, product number: ECLIPSE E-200), and Acanthamoeba adhered within a 5×5 mm observation area on the test piece surface were counted. Subsequently, trypan blue was applied to the observation area, staining dead Acanthamoeba, and the number of dead Acanthamoeba was counted. The results are shown in the following Table 1 and FIGS. 2 and 3.

TABLE 1

| Lysozyme- | Lysozyme- | Total number | |
|---|---|---|---|
| chitosan complex | chitosan complex | of adhered | Acanthamoeba |
| concentration | concentration | Acanthamoeba | mortality |
| (μg/mL) | (mass %) | (count/mm²) | rate (%) |
| 0 | 0 | 124 | 3.2 |
| 0.1 | 0.00001 | 108 | 3.5 |
| 1 | 0.0001 | 21.4 | 3.7 |
| 10 | 0.001 | 18.9 | 5.6 |
| 100 | 0.01 | 1.5 | 95.6 |
| 1000 | 0.1 | 1.3 | 99.4 |

As shown in Table 1, the lysozyme-chitosan complex induced cell death of Acanthamoeba in a concentration-dependent manner. The inhibitory effect of the lysozyme-chitosan complex on Acanthamoeba adhesion was observed from 0.1 μg/mL, with particularly notable adhesion inhibition effects observed at concentrations of 1 μg/mL and above. At 100 μg/mL and 1000 μg/mL, Acanthamoeba adhesion was almost completely inhibited, demonstrating excellent adhesion inhibition effects. Regarding the inhibitory effect (antiprotozoal effect) of the lysozyme-chitosan complex on Acanthamoeba, examining the number of dead Acanthamoeba and Acanthamoeba mortality rate in Table 1 shows that nearly 100% mortality rates were achieved particularly at 100 μg/mL and 1000 μg/mL, indicating significant antiprotozoal action.

### Experimental Example 2 (Comparative Adhesion Test and Cell Death Test)

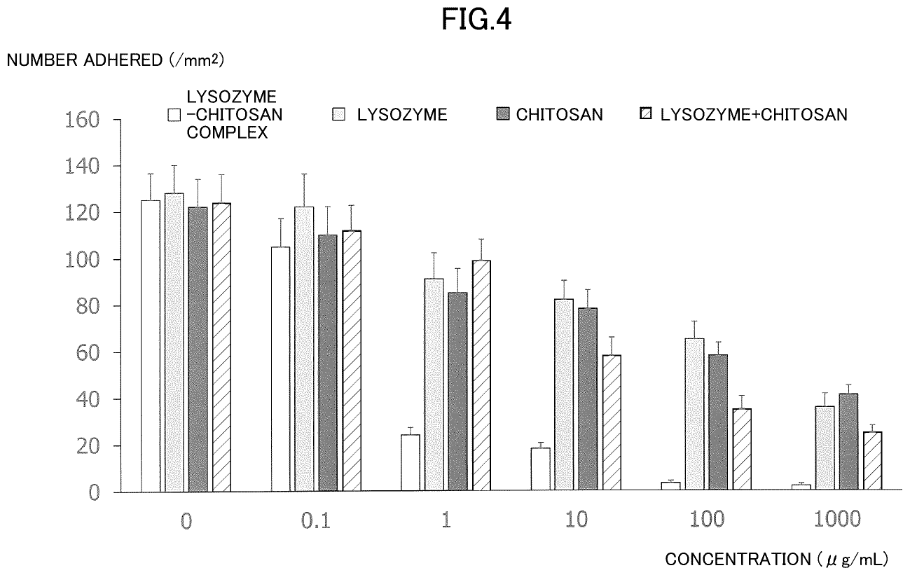

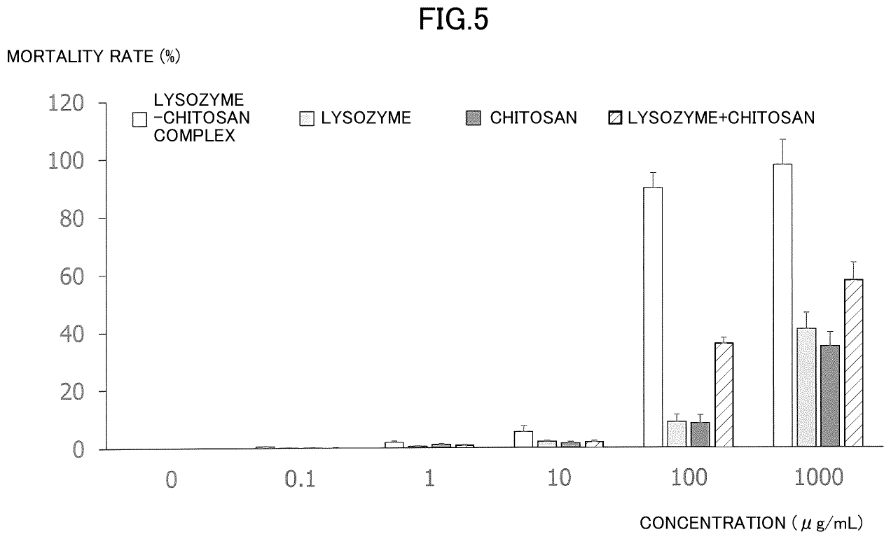

Acanthamoeba were counted and the total number of adhered Acanthamoeba, number of dead Acanthamoeba, and Acanthamoeba mortality rate were measured in the same manner as Experimental Example 1, except that the mixture solution of lysozyme and chitosan (Table 2), lysozyme alone (Table 3), and chitosan alone (Table 4) described above were used instead of the lysozyme-chitosan complex (LYZOX (registered trademark)) solution of Experimental Example 1. The results are shown in the following Tables 2-4 and FIGS. 4 and 5.

TABLE 2

| Mixture of lysozyme | Total number of | Acanthamoeba |
|---|---|---|
| and chitosan | adhered Acanthamoeba | mortality rate |
| (μg/mL) | (count/mm²) | (%) |
| 0 | 125 | 3.1 |
| 0.1 | 117 | 3.5 |
| 1 | 104 | 3.8 |
| 10 | 58.4 | 7.4 |
| 100 | 35.6 | 35.3 |
| 1000 | 29.4 | 62.1 |

TABLE 3

| | Total number of | Acanthamoeba |
|---|---|---|
| Lysozyme | adhered Acanthamoeba | mortality |
| (μg/mL) | (count/mm²) | rate (%) |
| 0 | 128 | 3.3 |
| 0.05 | 119 | 3.5 |
| 0.5 | 94.2 | 3.9 |
| 5 | 87.2 | 4.2 |
| 50 | 67.8 | 9.6 |
| 500 | 42.1 | 43.3 |

TABLE 4

| | Total number of | Acanthamoeba |
|---|---|---|
| Chitosan | adhered Acanthamoeba | mortality |
| (μg/mL) | (count/mm2) | rate (%) |
| 0 | 122 | 3.2 |
| 0.05 | 108 | 3.5 |
| 0.5 | 89.2 | 3.8 |
| 5 | 82.1 | 3.9 |
| 50 | 65.3 | 7.9 |
| 500 | 49.7 | 37.4 |

As shown in Tables 1-4 and FIG. 4, the lysozyme-chitosan complex of the present invention demonstrated superior Acanthamoeba adhesion inhibition effects compared to a mixture of the same mass of lysozyme and chitosan, lysozyme alone in the same mass as the lysozyme in the lysozyme-chitosan complex, and chitosan alone in the same mass as the chitosan in the lysozyme-chitosan complex. Particularly notable adhesion inhibition effects compared to the lysozyme and chitosan mixture, lysozyme alone, and chitosan alone were observed at concentrations of 1 μg/mL and above. Furthermore, as shown in Tables 2-4 and FIG. 5, the lysozyme-chitosan complex demonstrated superior Acanthamoeba mortality rates, i.e., Acanthamoeba inhibition effects (antiprotozoal effects), compared to a mixture of the same mass of lysozyme and chitosan, lysozyme alone in the same mass as the lysozyme in the lysozyme-chitosan complex, and chitosan alone in the same mass as the chitosan in the lysozyme-chitosan complex. While the lysozyme-chitosan complex of the present invention achieved nearly 100% mortality rates particularly at 100 μg/mL and 1000 μg/mL, the lysozyme and chitosan mixture, lysozyme alone, and chitosan alone showed incomplete Acanthamoeba inhibition effects.

### Experimental Example 3 (Comparative Adhesion Test and Cell Death Test)

Adhesion tests and cell death tests were conducted to verify the effects, particularly synergistic anti-adhesive and antiprotozoal properties, when using the lysozyme-chitosan complex (LYZOX (registered trademark)) with additional active ingredients.

First, Acanthamoeba-containing contact lens storage solutions were prepared. Specifically, Acanthamoeba (Acanthamoeba polyphaga, obtained from American Type Culture Collection (ATCC), 30872™) was dissolved in each commercially available contact lens storage solution listed in Table 5 to prepare Acanthamoeba-containing contact lens storage solutions containing 1×10⁶ Acanthamoeba per 1 mL of storage solution.

The above-mentioned lysozyme-chitosan complex (LYZOX (registered trademark)) solution was added to these prepared Acanthamoeba-containing contact lens storage solutions to prepare mixed solutions containing 100 μg (0.01 mass %) of lysozyme-chitosan complex per 1 mL of mixed solution. Separately, distilled water was used as a control. The same sterilized water used in preparing the lysozyme-chitosan complex was used as dilution liquid for concentration adjustment.

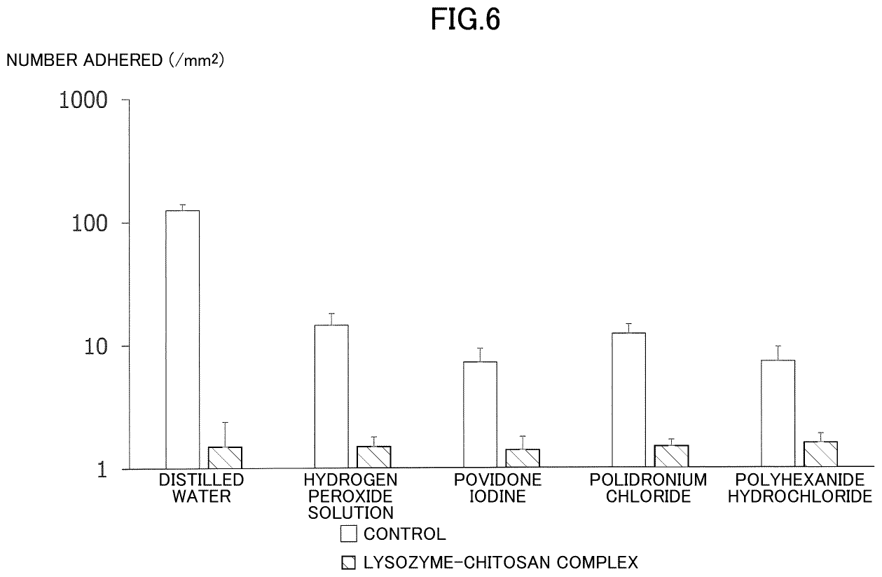

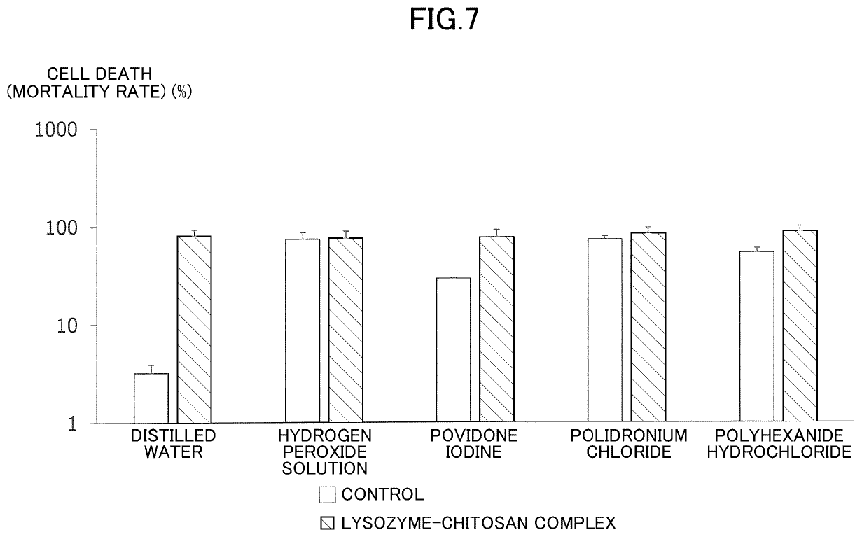

Polypropylene test pieces were immersed in the prepared mixed solutions, and adhered Acanthamoeba were counted and the number of dead Acanthamoeba was counted in the same manner as Experimental Example 1. The results are shown in the following Table 5 and FIGS. 6 and 7.

TABLE 5

| | | Concentration of | | Total number | |
|---|---|---|---|---|---|
| | | additional | | of adhered | Acanthamoeba |
| Contact lens | | active ingredient | | Acanthamoeba | mortality |
| storage solution | Additional active ingredient | (μg/mL (mass %)) | | (count/mm²) | rate (%) |
| Distilled water | — | | | 125 | 3.2 |
| | | | | 1.5 | 80.2 |
| ClearCare*¹ | Hydrogen peroxide solution | 30000 | μg/mL | 14.5 | 74.2 |
| | | (3%) | | 1.5 | 75.5 |
| Cleadew*¹ | Povidone iodine | 500 | μg/mL | 7.2 | 29.4 |
| | | (0.05%) | | 1.4 | 77.5 |
| Opti-Free*¹ | Polidronium chloride | 11 | μg/mL | 12.3 | 73.2 |
| | | (0.0011%) | | 1.5 | 84.5 |
| ReNu*¹ | Polyhexanide hydrochloride | 11 | μg/mL | 7.3 | 54.5 |

| | | |
|---|---|---|
| (0.0011%) | 1.6 | 89.0 |

* In Table 5, upper rows show storage solution only, lower rows show storage solution + lysozyme-chitosan complex
*¹ClearCare is a registered trademark, source: Alcon Japan Ltd., product name: AOSept ClearCare (registered trademark)
Cleadew is a registered trademark, source: Ophtecs Corporation, product name: Cleadew First Care (registered trademark)
Opti-Free is a registered trademark, source: Alcon Japan Ltd., product name: Opti-Free Plus (registered trademark)
ReNu is a registered trademark, source: Bausch & Lomb Japan Co., Ltd., product name: ReNu MultiPlus (registered trademark)

As shown in Table 5 and FIG. 6, while the adhesion inhibition effect of each contact lens storage solution alone was limited, notable adhesion inhibition effects were observed when combining contact lens storage solutions with the lysozyme-chitosan complex. Furthermore, regarding the inhibitory effect (antiprotozoal effect) of the lysozyme-chitosan complex on Acanthamoeba, examining the number of dead Acanthamoeba, Acanthamoeba mortality rate in Table 5, and FIG. 7 shows that good Acanthamoeba inhibition effects (antiprotozoal effects) were obtained when using the lysozyme-chitosan complex in combination with additional active ingredients, particularly povidone iodine and polyhexanide hydrochloride, compared to using contact lens storage solutions alone.

### INDUSTRIAL APPLICABILITY

The present invention can provide novel pharmaceutical compositions, antiprotozoal compositions, and anti-adhesion agents, etc., against Acanthamoeba.

1-8. (canceled)

9. An antiprotozoal composition comprising a complex of lysozyme and chitosan bound together.

10. The antiprotozoal composition according to claim 9, wherein the protozoan is Acanthamoeba.

11. (canceled)

12. A method of preventing adhesion of Acanthamoeba to a material surface, comprising applying a complex of lysozyme and chitosan bound together to the material surface.

13. The method for preventing adhesion of Acanthamoeba according to claim 12, wherein the material is selected from the group consisting of polyethylene, polypropylene, polyurethane, and polycarbonate.

14. The antiprotozoal composition according to claim 9, wherein the complex is contained in 0.0001 mass % to 5.0 mass % in the antiprotozoal composition.

15. The antiprotozoal composition according to claim 9, further comprising an additional active ingredient selected from the group consisting of hydrogen peroxide solution, povidone iodine, polidronium chloride, and polyhexanide hydrochloride.

16. The method for preventing adhesion of Acanthamoeba according to claim 12, wherein the material is polypropylene.

17. A method for treating, suppressing, or preventing inflammation caused by Acanthamoeba, comprising administering to a subject a pharmaceutical composition containing a complex of lysozyme and chitosan bound together.

18. The method according to claim 17, wherein the chitosan is a water-soluble chitosan having a molecular weight of 1000 Da to 30,000 Da.

19. The method according to claim 17, wherein the complex is contained in 0.01 μg/mL to 100,000 μg/mL in the pharmaceutical composition.

20. The method according to claim 17, wherein the complex is contained in 0.0001 mass % to 5.0 mass % in the pharmaceutical composition.

21. The method according to claim 17, further comprising an additional active ingredient selected from the group consisting of hydrogen peroxide solution, povidone iodine, polidronium chloride, and polyhexanide hydrochloride.

22. The method according to claim 17, wherein the inflammation caused by Acanthamoeba is dermatitis or keratitis.

23. The method according to claim 22, wherein the treatment or prevention of inflammation caused by Acanthamoeba is achieved through inhibition of Acanthamoeba adhesion.
